(19) United States
(12) Patent Application Publication
(10) Pub. No.: US 2005/0147576 A1
(43) Pub. Date: Jul. 7, 2005

(54) Composition containing an organopolysiloxane elastomer and an aminosulphonic compound

(75) Inventor: Thierry Cotton, Nice (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/028,229

(22) Filed: Jan. 4, 2005

(30) Foreign Application Priority Data
Jan. 6, 2004 (FR) 0450017

(51) Int. Cl. A61K 7/075; A61K 7/08

(52) U.S. Cl. 424/70.21

(57) ABSTRACT

The present invention relates to a composition including, in a physiologically acceptable medium, at least one organopolysiloxane elastomer and at least one aminosulphonic compound of formula: 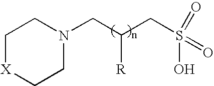 which is preferably 4-(2-hydroxyethyl)piperazine-1-ethanesulphonic acid (HEPES). It also relates to a cosmetic method for caring for the skin, intended in particular to tone down visible or tactile irregularities in the surface of the skin, in particular to tone down wrinkles and fine lines and/or skin blemishes, and/or to smooth the skin and/or to unify the complexion, comprising the topical application to the skin of the abovementioned composition.

### REFERENCE TO PRIOR APPLICATIONS

This application claims priority to French patent application 0450017 filed Jan. 6, 2004, both incorporated herein by reference.

### FIELD OF THE INVENTION

The present invention relates to a composition including, preferably in a physiologically acceptable medium, at least one organopolysiloxane elastomer and at least one given aminosulphonic compound.

It also relates to a cosmetic method for caring for the skin, intended in particular to tone down visible or tactile irregularities of the surface of the skin, in particular to tone down wrinkles and fine lines and/or skin blemishes, and/or to smooth the skin and/or to unify the complexion, comprising the topical application to the skin of the abovementioned composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The human skin is composed of two compartments, namely a surface compartment, the epidermis, and a deep compartment, the dermis.

The natural human epidermis is composed mainly of three types of cells which are the keratinocytes, which form the vast majority, the melanocytes and the Langerhans cells. Each of these cell types contributes via its specific functions to the essential role played by the skin in the body.

During the process known as keratinization, the keratinocytes situated in the basal layer of the epidermis multiply and grow, thus pushing the older epidermal cells upwards and towards the surface of the epidermis. During this displacement, these cells become flattened and differentiate to form keratin. The dead surface cells resulting from this keratinization process (corneocytes) constitute the horny layer of the epidermis, where they are separated by lipid layers and connected to one another by protein bonds (corneosomes). These dead cells are gradually removed from the surface of the skin and replaced by new keratinized cells.

In young healthy skin, the desquamation of the skin which thus occurs is characterized by the removal of individual cells or of small cell clusters. In contrast, with age or in the case of certain pathologies, the desquamation can be detrimentally affected, in the sense that an excess of keratinous substance is formed at the surface of the skin, resulting either in removal from the stratum corneum in the form of squamae (cutaneous ageing, dry skin) or in obstruction of the sebaceous follicles (acne).

Various agents intended to combat cutaneous ageing, particularly by promoting desquamation, that is to say removal of the “dead” cells situated at the surface of the horny layer of the epidermis, are known in the prior art. This “desquamating” property is also called, often wrongly, a keratolytic property.

Thus, Patent U.S. Pat. No. 4,603,146 discloses the use of retinoic acid and of its derivatives in cosmetic compositions for the purpose of combating cutaneous ageing.

Furthermore, numerous patents and publications (see, for example, Application EP-A-413 528), and numerous commercial cosmetic compositions, teach the use of α-hydroxy acids, such as lactic acid, glycolic acid or citric acid, for treating cutaneous ageing.

Finally, β-hydroxy acids, and more especially salicylic acid and its derivatives, are known for their desquamating properties (see the documents WO-A-93/10756 and U.S. Pat. No. 4,767,750).

Although their effectiveness is satisfactory, these desquamating compounds of the prior art can cause phenomena of irritation or of discomfort, in particular in subjects having a sensitive skin. Other desquamating compounds have thus been provided, for example by L'Oreal, in Application EP-1 337 233. This is a family of aminosulphonic derivatives which have the ability to decompose corneodesmosins (constituent proteins of the corneosomes) by promoting the activity of proteases (of chymotrypsin-like and trypsin-like type, in particular), while being well tolerated.

Due to their biological activity, these compounds do not have an immediate effect and the benefit visible on the skin of the epidermal renewal which they bring about is only obtained after using for some days, indeed even some weeks.

### SUMMARY OF THE INVENTION

The inventor has now discovered that, by combining an aminosulphonic derivative with an organopolysiloxane elastomer, the resultant composition significantly improves the surface quality of the skin right from the application and in a way prolonged over time.

### DETAILED DESCRIPTION OF THE PREFERRED EMBODIMENTS

One subject-matter of the present invention is thus a composition comprising, preferably in a physiologically acceptable medium, at least one organopolysiloxane elastomer and at least one aminosulphonic compound chosen from the compounds corresponding to the following formula (I): 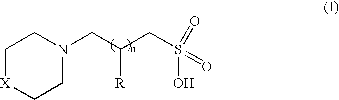
in which:

R denotes a hydrogen atom or a group chosen from —OH and —NH₂,

X denotes:

an oxygen atom,

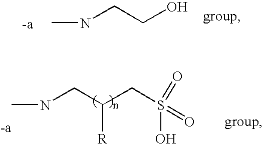

n is equal to 0, 1, 2 or 3,
and their physiologically acceptable salts. When X denotes 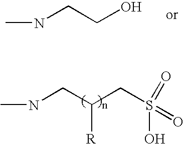
the nitrogen atom of these groups constitutes a part of the six membered ring.

The scope of the invention extends to the optical and/or geometrical isomers of the compounds of formula (I), alone or as a mixture in any proportions.

Mention may be made, among the compounds of formula (I) preferably used according to the invention, of:

4-(2-hydroxyethyl)piperazine-1-ethanesulphonic acid, which corresponds to the following formula:

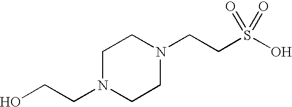

4-(2-hydroxyethyl)piperazine-1-(2-hydroxypropanesulphonic acid), which corresponds to the following formula:

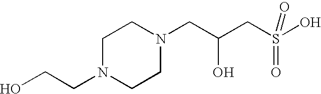

4-(2-hydroxyethyl)piperazine-1-propanesulphonic acid, which corresponds to the following formula:

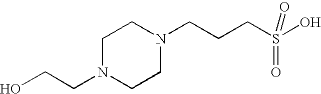

3-morpholinopropanesulphonic acid, which corresponds to the following formula:

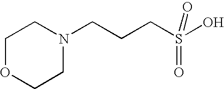

2-morpholinopropanesulphonic acid, which corresponds to the following formula:

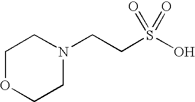

piperazine-1,4-bis(2-ethanesulphonic acid), which corresponds to the following formula:

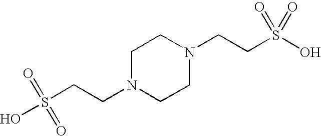

piperazine-1,4-bis(2-hydroxypropanesulphonic acid), which corresponds to the following formula:

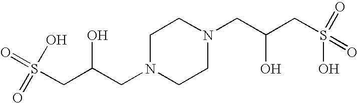

Preference is very particularly given, among these compounds, to 4-(2-hydroxyethyl)piperazine-1-ethanesulphonic acid or HEPES, sold in particular by Applichem.

The amount of aminosulphonic compound of formula (I) which can be used according to the invention is not particularly limited and can represent for example from 0.001 to 20%, preferably from 0.01 to 10% and better still from 0.1 to 5% of the total weight of the composition. As noted above, mixtures of compounds of formula (I) may be used in the invention.

The composition of the invention comprises at least one organopolysiloxane elastomer, which is preferably at least partially crosslinked. The term “elastomer” is understood to mean a flexible and deformable solid material having viscoelastic properties and in particular the consistency of a sponge or of a flexible sphere. Its modulus of elasticity is such that this material is resistant to deformation and has a limited ability to expand and to contract. This material is capable of returning to its original shape after it has been stretched. This elastomer is formed of polymeric chains of high molecular weight, the mobility of which is limited by a uniform network of crosslinking points.

The organopolysiloxane elastomers used in the composition according to the invention are preferably partially or completely crosslinked. They are preferably provided in the form of particles. In particular, the particles of organopolysiloxane elastomer preferably have a size ranging from 0.1 to 500 μm, preferably from 3 to 200 μm and better still from 3 to 50 μm. These particles can have any shape and, for example, can be spherical, flat or amorphous.

Crosslinked organopolysiloxane elastomers can be obtained by a crosslinking addition reaction of a diorganopolysiloxane comprising at least one hydrogen atom bonded to a silicon atom and a diorganopolysiloxane having at least two groups comprising ethylenic unsaturation bonded to separate silicon atoms, in particular in the presence of a platinum catalyst; or by a crosslinking condensation/dehydrogenation reaction between a diorganopolysiloxane comprising hydroxyl endings and a diorganopolysiloxane comprising at least one hydrogen atom bonded to a silicon atom, in particular in the presence of an organotin compound; or by a crosslinking condensation reaction of a diorganopolysiloxane comprising hydroxyl endings and of a hydrolysable organopolysilane; or by thermal crosslinking of an organopolysiloxane, in particular in the presence of an organoperoxide catalyst; or by crosslinking of an organopolysiloxane by high energy radiation, such as gamma rays, ultraviolet rays or an electron beam.

Preferably, the crosslinked organopolysiloxane elastomer is obtained by a crosslinking addition reaction (A) of a diorganopolysiloxane comprising at least one hydrogen atom bonded to a silicon atom and (B) of a diorganopolysiloxane having at least two groups comprising ethylenic unsaturation each bonded to a separate silicon atom, in particular in the presence (C) of a platinum catalyst, as, for example, disclosed in Application EP-A-295 886.

The compound (A) is in particular an organopolysiloxane having at least two hydrogen atoms bonded to separate silicon atoms in each molecule.

The compound (A) can exhibit any molecular structure, in particular a linear chain or branched chain structure or a cyclic structure. The compound (A) can have a viscosity of 25° C. ranging from 1 to 50 000 centistokes, in particular in order to be highly miscible with the compound (B).

The organic groups bonded to the silicon atoms of the compound (A) can be alkyl groups, such as methyl, ethyl, propyl, butyl or octyl; substituted alkyl groups, such as 2-phenylethyl, 2-phenylpropyl or 3,3,3-trifluoropropyl; aryl groups, such as phenyl, tolyl or xylyl; substituted aryl groups, such as phenylethyl; and substituted monovalent hydrocarbonaceous groups, such as an epoxy group, a carboxylate ester group or a mercapto group.

The compound (A) can thus be chosen from methylhydropolysiloxanes comprising trimethylsiloxy endings, dimethylsiloxane/methylhydrosiloxane copolymers comprising trimethylsiloxy endings or dimethylsiloxane/methylhydrosiloxane cyclic copolymers.

The compound (B) is advantageously a diorganopolysiloxane having at least two lower (for example C₂-C₄) alkenyl groups; the lower alkenyl group can be chosen from vinyl, allyl and propenyl groups. These lower alkenyl groups can be situated in any position on the organopolysiloxane molecule but are preferably situated at the ends of the organopolysiloxane molecule. The organopolysiloxane (B) can have a branched-chain, linear-chain, cyclic or network structure but the linear-chain structure is preferred. The compound (B) can have a viscosity ranging from the liquid state to the gum state. Preferably, the compound (B) has a viscosity of at least 100 centistokes at 25° C.

In addition to the abovementioned alkenyl groups, the other organic groups bonded to the silicon atoms in the compound (B) can be alkyl groups, such as methyl, ethyl, propyl, butyl or octyl; substituted alkyl groups, such as 2-phenylethyl, 2-phenylpropyl or 3,3,3-trifluoropropyl; aryl groups, such as phenyl, tolyl or xylyl; substituted aryl groups, such as phenylethyl; and substituted monovalent hydrocarbonaceous groups, such as an epoxy group, a carboxylate ester group or a mercapto group.

The organopolysiloxanes (B) can be chosen from methylvinylpolysiloxanes, methylvinylsiloxane/dimethylsiloxane copolymers, dimethylpolysiloxanes comprising dimethylvinylsiloxy endings, dimethylsiloxane/methylphenylsiloxane copolymers comprising dimethylvinylsiloxy endings, dimethylsiloxane/diphenylsiloxane/methylvinylsiloxane copolymers comprising dimethylvinylsiloxy endings, dimethylsiloxane/methylvinylsiloxane copolymers comprising trimethylsiloxy endings, dimethylsiloxane/methylphenylsiloxane/methylvinylsiloxane copolymers comprising trimethylsiloxy endings, methyl(3,3,3-trifluoropropyl)polysiloxanes comprising dimethylvinylsiloxy endings and dimethylsiloxane/methyl(3,3,3-trifluoropropyl)siloxane copolymers comprising dimethylvinylsiloxy endings.

In particular, the organopolysiloxane elastomer can be obtained by reaction of a dimethylpolysiloxane comprising dimethylvinylsiloxy endings and of a methylhydropolysiloxane comprising trimethylsiloxy endings in the presence of a platinum catalyst.

Advantageously, the sum of the number of ethylenic groups per molecule of the compound (B) and of the number of hydrogen atoms bonded to silicon atoms per molecule of the compound (A) is at least 5.

It is advantageous for the compound (A) to be added in an amount such that the molecular ratio of the total amount of hydrogen atoms bonded to silicon atoms in the compound (A) to the total amount of all the groups comprising ethylenic unsaturation in the compound (B) is within the range from 1.5/1 to 20.1.

The compound (C) is the catalyst of the crosslinking reaction and is in particular chloroplatinic acid, chloroplatinic acid/olefin complexes, chloroplatinic acid/alkenylsiloxane complexes, chloroplatinic acid/diketone complexes, platinum black and platinum-on-support.

The catalyst (C) is preferably added in a proportion of 0.1 to 1000 parts by weight, better still of 1 to 100 parts by weight, as platinum metal proper per 1000 parts by weight of the total amount of the compounds (A) and (B).

The elastomer obtained can be a nonemulsifying elastomer or an emulsifying elastomer.

The term “nonemulsifying” defines organopolysiloxane elastomers not comprising polyoxyalkylene units. The term “emulsifying” means crosslinked organopolysiloxane elastomers having at least one polyoxyalkylene, in particular polyoxyethylene or polyoxypropylene, unit.

The crosslinked organopolysiloxane elastomer particles can be conveyed in the form of a gel composed of an elastomeric organopolysiloxane included in at least one hydrocarbonaceous oil and/or one silicone oil. In these gels, the organopolysiloxane particles are often nonspherical particles.

The crosslinked organopolysiloxane elastomer particles can also be provided in the form of a powder, in particular in the form of a spherical powder.

Nonemulsifying elastomers are disclosed in particular in Patents U.S. Pat. No. 4,970,252, U.S. Pat. No. 4,987,169, U.S. Pat. No. 5,412,004, U.S. Pat. No. 5,654,362 and U.S. Pat. No. 5,760,116 and in Application JP-A-61-194009.

Use may be made, as nonemulsifying elastomers, of those sold under the names “KSG-6”, “KSG-15”, “KSG-16”, “KSG-18”, “KSG-31”, “KSG-32”, “KSG-33”, “KSG-41”, “KSG-42”, “KSG-43” and “KSG-44” by Shin-Etsu, “DC 9040”, “DC 9041”, “DC 9509”, “DC 9505” and “DC 9506” by Dow Corning, “Gransil” by Grant Industries and “SFE 839” by General Electric.

Advantageously, the emulsifying elastomers comprise the polyoxyalkylene-modified elastomers formed from divinyl compounds, in particular polysiloxanes having at least two vinyl groups, reacting with Si—H bonds of a polysiloxane. Emulsifying elastomers are disclosed in particular in Patents U.S. Pat. No. 5,236,986, U.S. Pat. No. 5,412,004, U.S. Pat. No. 5,837,793 and U.S. Pat. No. 5,811,487.

Use may be made, as emulsifying elastomers, of those sold under their names “KSG-21”, “KSG-20”, “KSG-30” and X-226146” by Shin-Etsu and “DC9010” and “DC9011” by Dow Corning.

The crosslinked organopolysiloxane elastomer can also be provided in the form of a crosslinked organopolysiloxane elastomer powder coated with silicone resin, in particular silsesquioxane resin, as disclosed, for example, in Patent U.S. Pat. No. 5,538,793. Such elastomers are sold under the names “KSP-100”, “KSP-101”, “KSP-102”, “KSP-103”, “KSP-104” and “KSP-105” by Shin-Etsu.

Other crosslinked organopolysiloxane elastomers in the form of powders can be powders formed of hybrid silicone functionalized by fluoroalkyl groups, sold in particular under the name “KSP-200” by Shin-Etsu; or powders formed of hybrid silicones functionalized by phenyl groups, sold in particular under the name “KSP-300” by Shin-Etsu.

Preferably, the organopolysiloxane elastomer used in the composition of the invention is capable of being obtained by hydrosilylation of polydimethylsiloxanes comprising end vinyl groups, comprising from 35 to 45 dimethylsiloxane units, by polymethylhydrosiloxane/dimethylsiloxanes comprising two methylhydrosiloxane units and from 25 to 35 and more preferably 30 dimethylsiloxane units (KSG-6 from Shin-Etsu).

The composition according to the invention includes, for example, from 0.1 to 30% by weight, better still from 0.5 to 10% by weight and even better still from 1 to 5% by weight of organopolysiloxane elastomer.

The composition according to the invention is preferably generally suited to topical application to the skin and thus preferably generally comprises a physiologically acceptable medium, that is to say a medium compatible with the skin and/or its superficial body growths. Thus, the composition according to the invention preferably has a pH of less than 8, better still of less than or equal to 7 and even better still of between 6 and 7.

The composition according to the invention can be provided in any form, including those conventionally used for topical application and in particular in the form of dispersions of the lotion or gel type, of emulsions with a liquid or semiliquid consistency of the milk type, obtained by dispersion of a fatty phase and an aqueous phase (O/W) or vice versa (W/O), or of suspensions or emulsions with a soft, semisolid or solid consistency of the cream or gel type, or of multiple emulsions (W/O/W or O/W/O), of microemulsions, of vesicular dispersions of ionic and/or nonionic type, or of wax/aqueous phase dispersions. These compositions can be prepared according to known methods.

According to a preferred embodiment of the invention, the composition is provided in the form of an O/W emulsion.

Mention may be made, as oils which can be used in the composition according to the invention, of:

hydrocarbonaceous oils of animal origin, such as perhyrosqualene;

hydrocarbonaceous oils of vegetable origin, such as vegetable oils, in particular the liquid fraction of karite butter;

synthetic esters and ethers, in particular of fatty acids, such as, for example, pentaerythritol esters, such as pentaerythrityl tetraethylhexanoate;

linear or branched hydrocarbons of mineral or synthetic origin, such as hydrogenated polyisobutene;

fatty alcohols having from 8 to 26 carbon atoms, such as cetyl alcohol, stearyl alcohol and their mixture, or octyldodecanol;

silicone oils, such as volatile or nonvolatile polymethylsiloxanes (PDMS) comprising a linear or cyclic silicone chain; and

their mixtures.

When it is present, the fatty phase of a composition according to the invention can comprise other fatty substances in addition to oils, such as: fatty acids comprising from 8 to 30 carbon atoms; waxes; and gums, such as silicone gums (dimethiconol).

These fatty substances can be chosen in a way varied by a person skilled in the art in order to prepare a composition having the desired properties, for example of consistency or of texture, in view of this disclosure.

This composition can additionally comprise various adjuvants such as those commonly used in the cosmetics field, such as emulsifiers, including glyceryl stearate and poly(ethylene glycol) stearate; fillers, in particular with a soft focus effect, such as colloidal silica dispersions, or block copolymers, such as disclosed in U.S. Pat. No. 6,013,682; preservatives; sequestering agents; colorants; fragrances; and thickening and gelling agents, in particular polyacrylamides and acrylic copolymers. The amount of these various adjuvants and their nature should preferably be chosen so as not to harm the properties of the composition according to the invention.

According to a preferred embodiment, the composition according to the invention additionally comprises at least one hydroxy acid and/or at least one heterogeneous polysaccharide.

Mention may be made, as hydroxy acids, of α-hydroxy acids, such as citric acid, lactic acid, glycolic acid, tartaric acid, mandelic acid and/or malic acid, and β-hydroxy acids, such as salicylic acid and its derivatives, in particular 5-(n-octanoyl)salicylic acid.

Use is preferably made, according to the invention, of a combination of 5-(n-octanoyl)salicylic acid and of glycolic acid.

The hydroxy acids can represent for example from 0.001 to 20%, preferably from 0.01 to 10% and better still from 0.1 to 1% of the total weight of the composition.

The term “heterogeneous polysaccharide” is understood to mean, according to the present invention, polymers composed of a combination of different monosaccharides or of monosaccharides having the same empirical chemical formula but with a different geometrical configuration (D and L isomers, for example). These polymers are distinguished both from polyheterosides, which are composed of one or more monosaccharides and of a non-glucide part, and from homogeneous polysaccharides, which result from the combination of the same monosaccharide. Thus, when it is present in the composition according to the invention, the heterogeneous polysaccharide is composed solely of sugars and results from the combination of at least two different monosaccharides.

The polysaccharides used in the composition according to the invention can be composed of 2 to 10 monosaccharides, compounds commonly referred to as oligosaccharides, or more than 10 monosaccharides, compounds commonly referred to as polysaccharides.

The monosaccharides present in the polysaccharide according to the invention can be chosen from any monosaccharide of natural or synthetic origin which can be envisaged and in particular such as:

aldoses, for example

pentoses: ribose, arabinose, xylose or apiose, for example,

hexoses: glucose, fucose, mannose or galactose, for example,

ketoses, such as fructose,

deoxymonosaccharides, such as rhamnose, digitoxose, cymarose or oleandrose,

monosaccharide derivatives, such as uronic acids, for example mannuronic acid, guluronic acid, galacturonic acid or glycuronic acid, or itols, such as mannitol or sorbitol.

In the context of the present invention, use may be made of a heterogeneous polysaccharide alone or of a mixture of heterogeneous polysaccharides.

Preferably, the heterogeneous polysaccharide comprises at least one fucose unit which can be present in an amount of 10-90% by weight, preferably 15-35% by weight, with respect to the weight of polysaccharide dry matter.

In particular, the polysaccharide according to the invention can comprise fucose, galactose and galacturonic acid units and, for example, can comprise a linear sequence of α-L-fucose, of α-D-galactose and of galacturonic acid. Such a polysaccharide is available in particular from Solabia under the trade name Fucogel 1000 PP®.

The polysaccharides according to the invention are preferably introduced into the composition in the form of an aqueous solution which can comprise 0.1 to 5% by weight of polysaccharide.

The polysaccharide can be present in the final composition for example in an amount of 0.001 to 50% by weight, preferably of 0.01 to 10% by weight and better still of 0.01 to 0.1% by weight.

To strengthen the effects of the composition according to the invention, the latter can furthermore include at least one compound chosen from: moisturizing agents, such as ceramides, hyaluronic acid and, in particular, glycerol; depigmenting agents, such as vitamin C and its derivatives; agents which stimulate the differentiation of keratinocytes; and agents for combating pollution and/or free radicals, in particular tocopherol and its esters.

The composition according to the invention can additionally comprise at least one UV-A and/or UV-B screening agent. The sunscreen agents can be chosen from organic screening agents, inorganic screening agent and their mixtures.

The more particularly preferred organic screening agents are chosen from the following compounds: ethylhexyl salicylate, ethylhexyl methoxycinnamate, octocrylene, phenylbenzimidazole sulphonic acid, benzophenone-3, benzophenone-4, benzophenone-5, 4-methylbenzylidene camphor, terephthalylidene dicamphor sulphonic acid, 2,4,6-tris(diisobutyl 4′-aminobenzalmalonate)-s-triazine, anisotriazine, ethylhexyl triazone, diethylhexyl butamido triazone, methylene bis-benzotriazolyl tetramethylbutylphenol, drometrizole trisiloxane, the derivatives of 4,4-diarylbutadiene, and their mixtures.

The inorganic screening agents are chosen from pigments or nanopigments (mean size of the primary particles, generally between 5 nm and 100 nm, preferably between 10 nm and 50 nm) formed of coated or uncoated metal oxides, such as, for example, titanium oxide (amorphous or crystalline in the rutile and/or anatase form), iron ozide, zinc oxide, zirconium oxide or cerium oxide nanopigments.

The composition according to the invention makes it possible to soften, without discomfort, visible and tactile irregularities of the skin surface.

Another subject-matter of the invention is thus a cosmetic process of caring for the skin, in particular for toning down visible or tactile irregularities of the surface of the skin, in particular for toning down wrinkles and fine lines and/or cutaneous blemishes, and/or smoothing the skin and/or unifying the complexion, comprising the topical application to the skin of the abovementioned composition.

The invention will now be illustrated by the following nonlimiting examples. In these examples, the amounts are indicated as percentage by weight.

### EXAMPLES

### Example 1

Cream for Dry Skin

An O/W emulsion having the composition below is prepared in a way conventional to a person skilled in the art.

| | |
|---|---|
| Glycolic acid | 0.05% |
| 5-(n-octanoyl)salicylic acid | 0.20% |
| Heterogeneous polysaccharide at 1% in water | 5.00% |
| (Fucogel 1000 PP from Solabia) | |
| Hydroxyethylpiperazineethanesulphonic acid | 1.00% |
| Alcohol | 2.00% |
| Glycerol | 3.00% |
| Tocopherol at 50% in soybean oil | 0.10% |
| Glyceryl stearate | 0.50% |
| Silicone elastomer in oil | 2.60% |
| (KSG-6 from Shin-Etsu) | |
| Disodium EDTA | 0.05% |
| Oils | 25.00% |
| Fillers | 2.50% |
| Thickening polymers | 2.00% |
| Preservatives | q.s. |
| Neutralizing agent q.s. | pH = 6.5 |
| Fragrance | q.s. |
| Waterq.s. for | 100.00% |

This composition can be applied in the morning and/or evening to the face to moisturize and smooth the skin.

### Example 2

Cream for Normal to Combination Skin

An O/W emulsion having the composition below is prepared in a way conventional to a person skilled in the art.

| | |
|---|---|
| Lactic acid | 0.05% |
| Salicylic acid | 0.30% |
| Heterogeneous polysaccharide at 1% in water | 5.00% |
| (Fucogel 1000 PP from Solabia) | |
| Hydroxyethylpiperazineethanesulphonic acid | 1.00% |
| Alcohol | 4.00% |
| Glycerol | 3.00% |
| Tocopherol at 50% in soybean oil | 0.10% |
| Glyceryl stearate | 0.50% |
| Silicone elastomer in oil | 2.60% |
| (KSG-6 from Shin-Etsu) | |
| Disodium EDTA | 0.05% |
| Oils | 20.00% |
| Fillers | 2.50% |
| Thickening polymers | 2.00% |
| Preservatives | q.s. |
| Neutralizing agent q.s. | pH = 6.5 |
| Fragrance | q.s. |
| Water q.s. for | 100.00% |

This composition can be applied in the morning and/or evening to the face to smooth and refine the skin grain and to unify the complexion.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a physiologically acceptable medium, at least one organopolysiloxane elastomer and at least one aminosulphonic compound selected from the group consisting of compounds corresponding to the following formula (I): 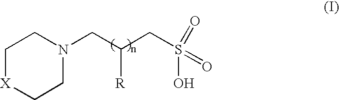
in which:
R denotes a hydrogen atom or a group chosen from —OH and —NH₂,
X denotes:
an oxygen atom, 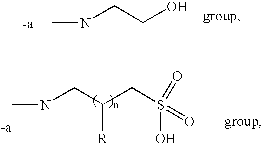
n is equal to 0, 1, 2 or 3,
their physiologically acceptable salts, and mixtures thereof.

2. The composition according to claim 1, wherein the organopolysiloxane is partially or completely crosslinked.

3. The composition according to claim 2, wherein the crosslinked organopolysiloxane elastomer is obtained by a crosslinking addition reaction (A) of a diorganopolysiloxane comprising at least one hydrogen atom bonded to a silicon atom and (B) of a diorganopolysiloxane having at least two groups comprising ethylenic unsaturation each bonded to a separate silicon atom.

4. The composition according to claim 3, wherein the compound (A) is selected from the group consisting of methylhydropolysiloxanes comprising trimethylsiloxy endings, dimethylsiloxane/methylhydrosiloxane copolymers comprising trimethylsiloxy endings dimethylsiloxane/methylhydrosiloxane cyclic copolymers, and mixtures thereof.

5. The composition according to claim 3, wherein the organopolysiloxane (B) is selected from the group consisting of methylvinylpolysiloxanes, methylvinylsiloxane/dimethylsiloxane copolymers, dimethylpolysiloxanes comprising dimethylvinylsiloxy endings, dimethylsiloxane/methylphenylsiloxane copolymers comprising dimethylvinylsiloxy endings, dimethylsiloxane/diphenylsiloxane/methylvinylsiloxane copolymers comprising dimethylvinylsiloxy endings, dimethylsiloxane/methylvinylsiloxane copolymers comprising trimethylsiloxy endings, dimethylsiloxane/methylphenylsiloxane/methylvinylsiloxane copolymers comprising trimethylsiloxy endings, methyl(3,3,3-trifluoropropyl)polysiloxanes comprising dimethylvinylsiloxy endings, dimethylsiloxane/methyl(3,3,3-trifluoropropyl)siloxane copolymers comprising dimethylvinylsiloxy endings, and mixtures thereof.

6. The composition according to claim 2, wherein the organopolysiloxane elastomer is obtained by reaction of a dimethylpolysiloxane comprising dimethylvinylsiloxy endings and of a methylhydropolysiloxane comprising trimethylsiloxy endings in the presence of a platinum catalyst.

7. The composition according to claim 6, wherein the organopolysiloxane elastomer is obtained by hydrosilylation of polydimethylsiloxanes comprising end vinyl groups, comprising from 35 to 45 dimethylsiloxane units, by polymethylhydrosiloxane/dimethylsiloxanes comprising two methylhydrosiloxane units and from 25 to 35 dimethylsiloxane units.

8. The composition according to claim 7, wherein the polymethylhydrosiloxane/dimethylsiloxane comprises 30 dimethylsiloxane units.

9. The composition according to claim 1, wherein the aminosulphonic compound is 4-(2-hydroxyethyl)piperazine-1-ethanesulphonic acid.

10. The composition according to claim 1, comprising from 1 to 5% by weight of organopolysiloxane.

11. The composition according to claim 1, comprising from 0.1 to 5% by weight of compound of formula (I).

12. The composition according to claim 1, further comprising at least one hydroxy acid and/or at least one heterogeneous polysaccharide.

13. The composition according to claim 12, comprising an hydroxy acid that is an β-hydroxy acid.

14. The composition according to claim 13, wherein the α-hydroxy acid is selected from the group consisting of citric acid, lactic acid, glycolic acid, tartaric acid, mandelic acid, malic acid, and mixtures thereof.

15. The composition according to claim 12, comprising an hydroxy acid that is a β-hydroxy acid.

16. The composition according to claim 15, wherein the β-hydroxy acid is selected from the group consisting of salicylic acid, 5-(n-octanoyl)salicylic acid, and mixtures thereof.

17. The composition according to claim 12, comprising a combination of 5-(n-octanoyl)salicylic acid and of glycolic acid.

18. The composition according to claim 12, comprising a polysaccharide that comprises a linear sequence of α-L-fucose, of α-D-galactose and of galacturonic acid.

19. The composition according to claim 1, wherein the composition is provided in the form of an O/W emulsion.

20. A process, comprising topically applying the composition according to claim 1 to the skin.

21. The process according to claim 20, comprising applying an amount of the composition according to claim 1 effective to tone down visible or tactile irregularities of the surface of the skin.

22. The process according to claim 20, comprising applying an amount of the composition according to claim 1 effective to tone down wrinkles and fine lines and/or cutaneous blemishes, and/or smooth the skin and/or unify the complexion.
